(19) United States
(12) Patent Application Publication
(10) Pub. No.: US 2025/0041039 A1
(43) Pub. Date: Feb. 6, 2025

(54) Biological Construct Delivery System

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 18/788,780

(22) Filed: Jul. 30, 2024

Related U.S. Application Data
(60) Provisional application No. 63/517,761, filed on Aug. 4, 2023.

(51) Int. Cl. A61F 2/00

(52) U.S. Cl. CPC A61F 2/0063; A61F 2002/0072; A61F 2230/0004

(57) ABSTRACT

A delivery system for biological constructs to targeted treatment sites within a patient body. The delivery system includes a passer or grasper that supports a construct or patch on the end of the grasper between a gripping pad. The passer includes forceps or arms that releasably contain the construct between the arm and the gripper pad. The passer advances the construct through a slotted cone that rolls up the construct as it is advanced through a cone and then exits the slotted cone via an insertion tip that passes the implant or construct through a cannula to a desired surgical site.

This application claims priority to U.S. Provisional Application 63/517,761 filed Aug. 4, 2023.

### FIELD OF THE INVENTIONS

The inventions described below relate to the field of delivery devices of biological constructs to targeted treatment sites within a patient body.

### BACKGROUND OF THE INVENTIONS

Endoscopic procedures that allow delivery of biological constructs offer significant advantages relative to conventional surgical procedures. Positioning and placement of biological constructs during such procedures can be inaccurate of difficult to execute. Accurate delivery of biological constructs to advantageous placement over desired tissue tear facilitates healing of the torn tissue.

### SUMMARY

The devices and methods described below provide for improved placement of biological constructs during endoscopic procedures by affording release and re-grasp or reposition of the biological construct when within a patient. The delivery system includes a passer or grasper that supports a construct or patch on the end of the grasper between a gripping pad. The passer includes forceps or arms that releasably contain the construct between the arm and the gripper pad. The passer advances the construct through a slotted cone that rolls up the construct as it is advanced through a cone and then exits the slotted cone via an insertion tip that passes the implant or construct through a cannula to a desired surgical site. The biological construct delivery system provides for improved introduction and positioning of the patch or construct into a desired surgical space. The biological construct delivery system includes a passer that can release and re-grab the biological construct for better handling and ease of use.

### BRIEF DESCRIPTION OF THE DRAWINGS

FIG. 1 illustrates a biological construct delivery system.

FIG. 2 illustrates details of the distal end of the passer or grasper of the biological construct delivery system.

FIG. 3 illustrates a passer of a biological construct delivery system with an alternative handle.

FIG. 4 illustrates the passer of the biological construct delivery system with a gripping pad attached to the passer arm.

FIG. 5 illustrates a top view of the passer of the biological construct delivery system.

FIG. 6 illustrates a side view of the passer of the biological construct delivery system.

FIGS. 7, 8 and 9 illustrates various spreader pad sheets for use with the passer of the construct delivery system.

FIGS. 10 and 11 illustrate a passer that includes a gripping pad with a flat or crochet needle stitcher.

### DETAILED DESCRIPTION OF THE INVENTIONS

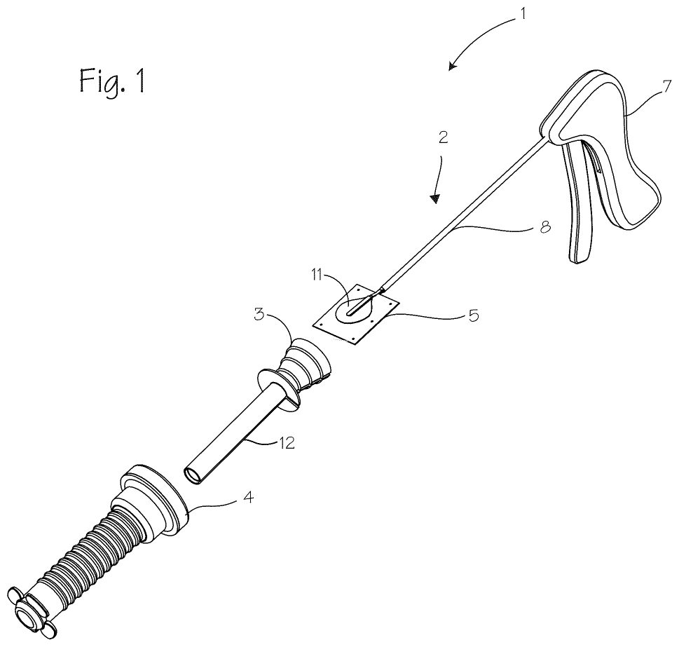

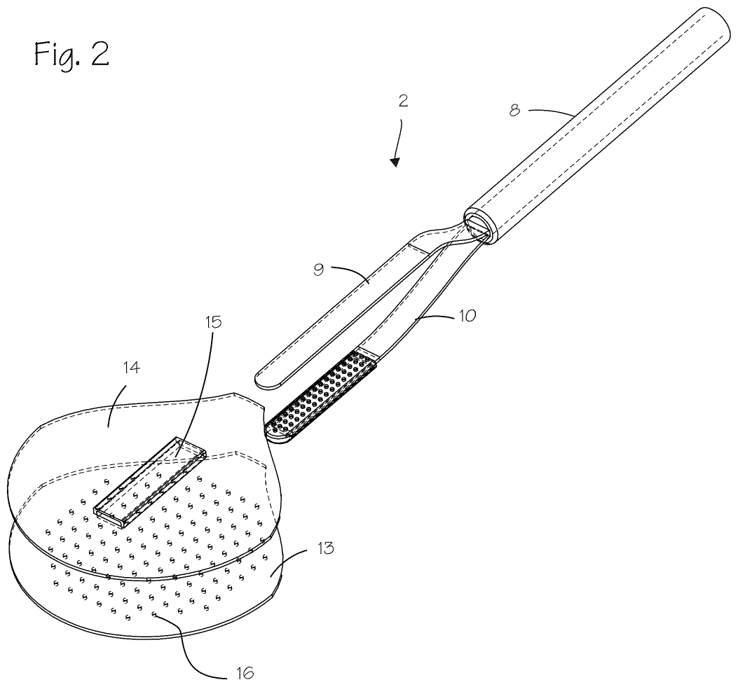

FIG. 1 illustrates a biological construct delivery system 1. The system includes a passer 2, a delivery cone 3 and obtutator, a cannula 4 and a biological construct sheet 5 positioned within the passer. The passer or grasper 6 includes a handle 7 and an attachment rod 8 extending from the handle. The passer has a proximal end and a distal end. The attachment rod extends longitudinally from the handle and is connected to a first arm 9 and a second arm 10 (shown in FIG. 2) at the distal end of the attachment rod. The passer further includes a gripping or spreader pad 11 having a first or top sheet and a second or bottom sheet. The spreader pad first sheet releasably attaches to an arm of the passer. A biological construct sheet 5 is releasably secured between the first and second sheets of the passer and then positioned between the passer first and second arms for delivery to a desired surgical space within a patient. The delivery cone 3 has a proximal end and a distal end. The cone includes a longitudinally transverse slot 12. The cone is open at the proximal end and tapers toward the distal end in a cone or funnel shape. The biological construct delivery system further includes a cannula 4. The delivery cone is inserted into the cannula in an assembled position.

FIG. 2 illustrates details of the distal end of the passer or grasper 6 of the biological construct delivery system. The passer is a tool used to guide and place a biological construct onto a desired treatment site. The passer includes a handle 7 (shown in FIG. 1) and an attachment rod 8 that extends longitudinally from the handle. The distal end of the attachment rod includes a first arm 9 and a second arm 10. The first and second arms are made of a resilient material such as mylar and are operable to spring open and closed. The first and second arms secure the spreader pad 11 that includes a first resilient polymer sheet 13 and a second resilient polymer sheet 14. The spreader pad sheets assist in securing a biological construct or patch between the spreader pads. The gripping pads releasably secure the implant once in position to ensure that it does not slide around on the arms when rolled for insertion through the cone for introduction into a patient. The gripping pad locks also holds the biological construct or implant securely between the passer springy arms once in a preferred position. However, spreader pad with its resilient polymer sheets also allows for release and re-grab of the patch to allow repositioning of a biological construct before final placement by allowing the resilient polymer sheets to open/close and also move biological construct within surgical area.

The spreader pad is illustrated in layers. A first or top sheet 13 is a resilient polymer layer. For example, the first sheet can be made of mylar (PETG) and be between 1 and 15 mils thick. The first sheet can also include an attachment feature 15 to attach the spreader pad to the first arm of the grasper. The spreader pad also includes a second or bottom sheet 14. The second sheet can be made of any elastomer material such as bondable soft TPE, silicone, Mylar, PET or spring memory polymer. The first and second sheets can be bonded to each other via a biocompatible adhesive between the sheets. Both the first sheet and the second sheet can include at least one surface that has a textured grip 16 to better hold the biologic construct. The grip can be made of urethane that can include molded texture within the polymer sheet surface. The textured urethane grip layer can be laminated to the polymer payer of the grip pads. The first and second spreader pad sheets can be any shape, but are preferably the same shape. The resilient polymer spreader pad allows for repositioning of the biological construct. The spreader pad securely maintains the biological construct when advancing to desired treatment area. But once in the desired location, the metal clamp arms can be manipulated to open and close to releasably reposition the biological construct from the desired treatment site until the construct is in the correct position before stapled to the treatment site.

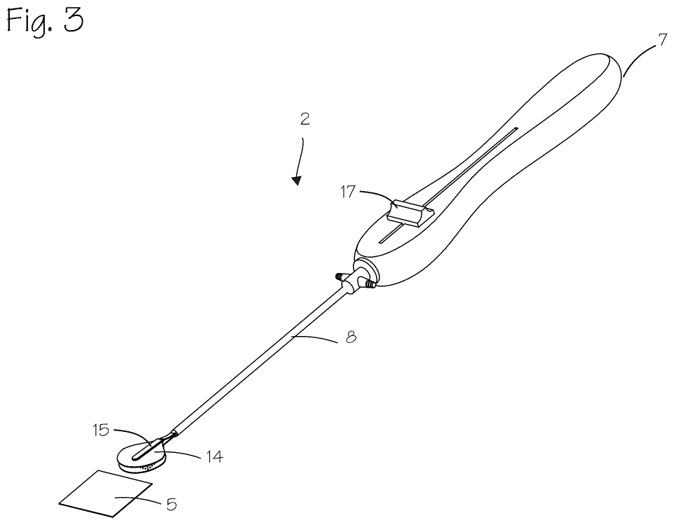

FIG. 3 illustrates a passer of a biological construct delivery system with an alternative handle. The passer includes a flat slider grip handle. The handle includes an open/close slider 17 to open and close the arms at the distal end of The elongate rod contains a first arm and a second arm that are operated by the open close slider at the distal end of the passer. The arms are connected to the spreader pad and contain a biological construct between the first sheet and the second sheet of the spreader pad.

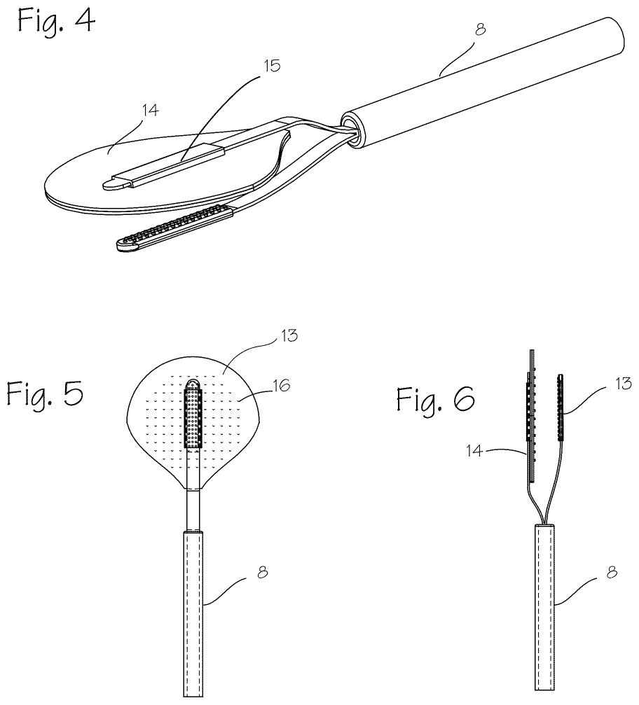

FIG. 4 illustrates the passer 6 of the biological construct delivery system with the first or top gripping pad attached to a passer arm. A gripping pad is attached to the first arm of the passer via the attachment feature 15.

FIG. 5 illustrates a top view of the passer of the biological construct delivery system. It illustrates a gripping pad with a tapered proximal end secured to a passer arm that facilitates advancement and removal of the implant through the cannula.

FIG. 6 illustrates a side view of the passer of the biological construct delivery system. It illustrates the first arm and the second arm in an open position with the resilient polymer sheets attached to the arms.

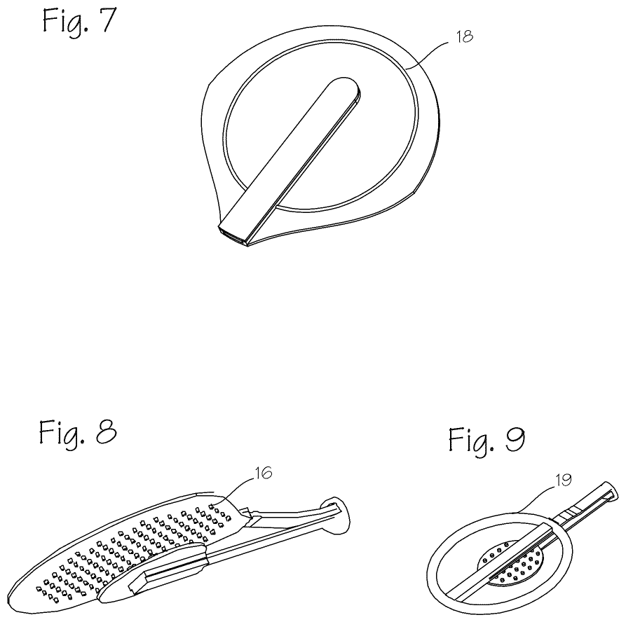

FIGS. 7, 8 and 9 illustrate various spreader pad sheets for use with the passer of the construct delivery system. FIG. 7 illustrates a spreader pad made of a resilient material such a silicone or TPE, over molded onto a nitinol wire ring 18. FIG. 8 illustrates a spreader pad with traction features in the form of molded protrusions that create a textured grip 16 spaced at predetermined intervals on the surface of the grip pad surface. FIG. 9 illustrates a spreader pad that includes an O-ring 19 with a central grip area instead of a flat sheet. All of these cause the sheet implants to roll into a flat position easily.

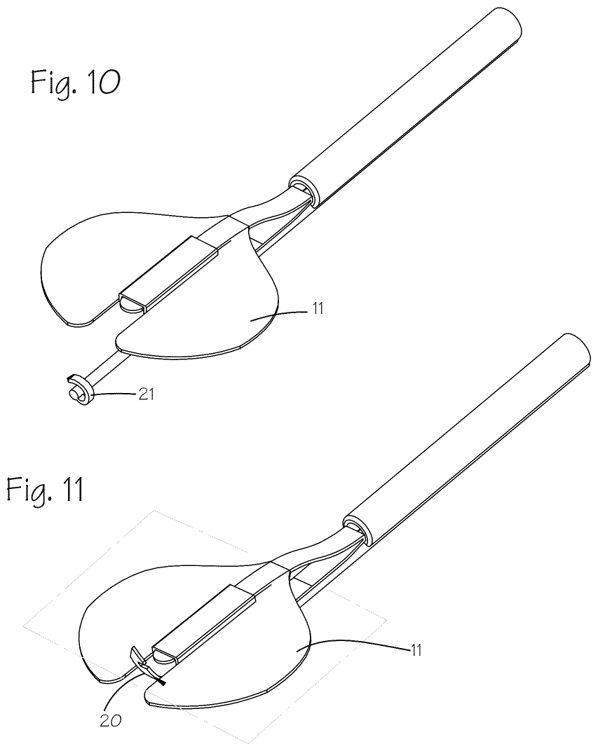

FIGS. 10 and 11 illustrate a passer that includes a gripping or spreader pad 11 with a flat needle 20 or crochet needle 21 stitcher. The passers include a stitcher with the sheet passer to attach the biological path or construct to a tendon. FIG. 10 is a crochet hook needle that loops a suture into body tissue from the top/bursal side. FIG. 11 illustrates a stitcher with a sheet passer and a flat needle that passes a suture from the underside/articular side of the rotator cuff tendon.

In use, a surgical incision is made to create access to a target tissue area. The surgical space can be created percutaneously with a dissector or distended with saline. A cannula is inserted into the surgical space. A delivery cone and obturator are inserted into the cannula. The obturator is then removed from the cannula. The arms of the passer are engaged into an open position and a biological construct sheet is placed between the open arms. The arms are then closed to secure the biological construct therebetween. The biological sheet construct is gripped between the arms of the passer and clamped so that the gripping pad of the biological sheet construct is attached to an arm of the spreader pad. The arms clamp the middle of the biological construct and form a pole for the biological sheet construct to wrap around as it is passed through the slotted delivery cone. The secured sheet is then placed within the delivery cone to roll for insertion. The arms of the passer are introduced into the slotted delivery cone and as they advance through, the delivery tunnel progressively rolls the biological construct until it is entirely rolled within the delivery cone and then pushed through the delivery cone, through the cannula, and then advanced into the surgical space. The slotted delivery cone is then pushed back out of the cannula and removed by passing it over the attachment rod of the passer via the slot on the delivery cone. The passer can be removed through the cannula, which is advantageous over prior systems. The slotted funnel may be used with a second cannula, or as a removable percutaneous cannula without a second cannula. Remove the slotted delivery cone from cannula over the passer. The passer arms continue to securely grip the construct or patch so that the surgeon maintains control of the sheet and can reposition it while preparing for fixation. The pad has a traction surface that gently but securely holds the biological construct. The pad is resilient and springy and lies flat, thus flattening out the biological construct sheet and preparing it for stapling to the patient. Position unrolled biological sheet construct on tendon and apply fixation. Unclamp passer and slide to new position and then reclamp. The surgeon can release and regrip the sheet for maximum control of the biological sheet during the positioning and fixation process. Apply additional fixation as needed. Open passer, slide away from fixated biological construct. Close and lock passer and then remove through the cannula.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A biological construct delivery system comprising:
a passer having a proximal end and a distal end, the passer having a handle and an elongated rod extending longitudinally from the handle and connected to a first arm and a second arm projecting from the elongated rod from the distal end of the passer;
a spreader pad having a first sheet and a second sheet, the spreader first sheet adapted for releasable attachment to the first arm of the passer and the second sheet releasably attached to the second arm of the passer;
a delivery cone with a proximal end and a distal end and a longitudinally traverse slot along the delivery cone, the proximal end of the delivery cone shaped for introduction of the biological construct into the delivery cone; and
a cannula releasably attached at distal end of delivery cone wherein the delivery cone is shaped for introduction into the cannula;
wherein the biological construct is releasably secured within the first sheet and the second sheet of the spreader pad.

2. The biological construct delivery system of claim 1 wherein the first and second sheets of the spreader pad are made of mylar.

3. The biological construct delivery system of claim 1 wherein the first and second sheets of the spreader pad are between 1 and 15 mils thick.

4. The biological construct delivery system of claim 2 wherein the first and second sheets of the spreader pad further compromise a textured urethane grip layer laminated to a surface of each sheet.

5. The biological construct delivery system of claim 1 wherein the first and second sheets of the spreader pad are the same size.

6. The biological construct delivery system of claim 1 further including an obturator configured to be inserted into the delivery cone.

7. The biological construct delivery system of claim 1 wherein the handle of the passer further comprises an open/close slider that is operable to open and close the passer arms.

8. A passer for use with a biological construct system comprising:
a handle;
an elongate rod that extends longitudinally from the handle and has first arm and second arm at an end of the elongate rod; and
a spreader pad including a first sheet and a second sheet, the spreader first sheet adapted for releasable attachment to the first arm of the passer and the second sheet releasably attached to the second arm of the passer.

9. The passer of claim 8 wherein the first and second sheets of the spreader pad are made of mylar.

10. The passer of claim 9 wherein the first and second sheets of the spreader pad further compromise a textured urethane grip layer laminated to a surface of each sheet.

11. The passer of claim 8 wherein the first and second sheets of the spreader pad are the same size.

12. The biological construct delivery system of claim 1 wherein the first and second sheets of the spreader pad are between 1 and 15 mils thick.

13. The passer of claim 8 wherein the handle of the passer further comprises an open/close slider that is operable to open and close the passer arms.

14. A method of delivering a biological construct to a target delivery area comprising the steps of:
creating access to a target tissue area;
inserting a cannula into the surgical space, then inserting a delivery cone and obturator into the cannula and then removing the obturator when the delivery cone is at the target location;
providing a passer comprising,
a handle;
an elongate rod that extends longitudinally from the handle and has first arm and second arm at an end of the elongate rod; and
a spreader pad including a first sheet and a second sheet, the spreader first sheet adapted for releasable attachment to the first arm of the passer and the second sheet releasably attached to the second arm of the passer;
placing the biological construct within spreader pad;
placing the passer with biological construct within a delivery cone and rolling the biological construct through delivery cone by advancing the passer through the delivery cone, through the cannula, and then advanced into the surgical space;
opening and closing the passer arms to reposition the biological construct until it is in a desired position;
applying fixation of the biological construct to the desired location;
open arms of the passer and slide the passer away from the fixated biological construct;
removing the passer through the cannula; and
removing the cannula.
